(19) United States
(12) Patent Application Publication
(10) Pub. No.: US 2009/0259299 A1
(43) Pub. Date: Oct. 15, 2009

(54) Side Branch Stent Having a Proximal Flexible Material Section

(75) Inventor: Noreen Moloney, Moycullen (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/102,182

(22) Filed: Apr. 14, 2008

(51) Int. Cl. A61F 2/82

(52) U.S. Cl. 623/1.35

(57) ABSTRACT

A stent system for placement in a bifurcated vessel includes a side branch stent. The side branch stent includes an expandable metallic distal section and a proximal section. The distal end of the proximal section is coupled to the distal section. The proximal section is made from a flexible material in the form of a sheet and is not supported by a frame proximally of the distal end. The proximal section includes a curled or tabbed proximal end adapted to engage walls of the main vessel at a junction of the main vessel and the side branch vessel. A second stent is configured for placement in the main vessel and the main vessel branch. Expansion of the second stent pushes the curled or tabbed proximal end of the first stent against the walls of the main vessel at the junction.

### FIELD OF THE INVENTION

The invention relates generally endoluminal prostheses, and more particularly to a stent intended for placement in a side branch vessel associated with a bifurcated or trifurcated vessel.

### BACKGROUND OF THE INVENTION

Heart disease, specifically coronary artery disease, is a major cause of death, disability, and healthcare expense in the United States and other industrialized countries. A number of methods and devices for treating coronary heart disease have been developed, some of which are specifically designed to treat the complications resulting from atherosclerosis and other forms of coronary arterial narrowing.

One method for treating such conditions is percutaneous transluminal coronary angioplasty (PTCA). Generally, PTCA is a procedure that involves passing a balloon catheter over a guidewire to a stenosis with the aid of a guide catheter. The stenosis may be the result of a lesion such as a plaque or thrombus. The guidewire extends from a remote incision to the site of the stenosis, and typically across the lesion. The balloon catheter is passed over the guidewire, and ultimately positioned across the lesion. Once the balloon catheter is appropriately positioned across the lesion, e.g., under fluoroscopic guidance, the balloon is inflated to break-up the plaque of the stenosis to thereby increase the vessel cross-section. The balloon is then deflated and withdrawn over the guidewire into the guide catheter to be removed from the body of the patient. In many cases, a stent or other prosthesis must be implanted to provide permanent support for the vessel. Stents are typically constructed of a metal or polymer and are generally a hollow cylindrical shape. When such a device is to be implanted, a balloon catheter, typically carrying a stent on its balloon, is deployed to the site of the stenosis. The balloon and accompanying stent are positioned at the location of the stenosis, and the balloon is inflated to circumferentially expand and thereby implant the stent. Thereafter, the balloon is deflated and the catheter and the guidewire are withdrawn from the patient.

Although systems and techniques exist that work well in many cases, no technique is applicable to every case. For example, special methods exist for dilating lesions that occur in branched or bifurcated vessels. A bifurcation is an area of the vasculature where a main vessel is bifurcated into two or more branch vessels. It is not uncommon for stenotic lesions to form at such bifurcations. The stenotic lesions can affect only one of the vessels, i.e., either of the branch vessels or the main vessel, two of the vessels, or all three vessels.

Methods to treat bifurcated vessels seek to prevent the collapse or obstruction of the main and/or branch vessel(s) during the dilation of the vessel to be treated. Such methods include techniques for using double guidewires and sequential percutaneous transluminal coronary angioplasty (PTCA) with stenting or the “kissing balloon” and “kissing stent” techniques, which provide side branch protection. Administering PTCA and/or implanting a stent at a bifurcation in a body lumen poses further challenges for the effective treatment of stenoses in the lumen. For example, dilating a vessel at a bifurcation may cause narrowing of an adjacent branch of the vessel. In response to such a challenge, attempts to simultaneously dilate both branches of the bifurcated vessel have been pursued. These attempts include deploying more than one balloon, more than one prosthesis, a bifurcated prosthesis, or some combination of the foregoing. However, simultaneously deploying multiple and/or bifurcated balloons with or without endoluminal prostheses, hereinafter individually and collectively referred to as a bifurcated assembly, requires highly accurate placement of the assembly. Specifically, deploying a bifurcated assembly requires positioning a main body of the assembly within the trunk of the vessel adjacent the bifurcation, and then positioning the independent legs of the assembly into separately branching legs of the body lumen.

Implanting a stent at a bifurcation in a body lumen requires additional consideration of appropriate stent sizes due to the relative sizes of the main vessel and the branch vessels. Some branch vessels can have somewhat smaller diameter lumens than the main vessel from which they branch. In addition, some branch vessels can have lumens with somewhat different diameters from each other. Therefore, stents of different sizes may be needed for properly deploying a stent in each of the main and branch vessels. It would be desirable to allow a clinician to custom-select different combinations of stent sizes for deploying stents in main or branch vessels having different diameter lumens. Further, it would be desirable to allow for differential sizing of a side branch stent even after the main vessel stent is implanted.

Further, stent implantation may cause undesirable reactions such as restensosis, inflammation, infection, thrombosis, and proliferation of cell growth that occludes the passageway. These reactions are especially common when repairing a vessel affected by stenosis at the point at which the vessel originates, branching off from an adjoining vessel. This point of origin is referred to as the ostium of the vessel, which is prone to restenosis. A bulk of material (such as, for example, overlapping stent struts) often occurs at the ostium and acts as an initiation site for thrombus and/or restenosis. To assist in preventing these conditions, stents have been used with coatings to deliver drugs or other therapeutic agents at the site of the stent. However, it would be desirable to provide a side branch stent having a design or structure that allows for less turbulent blood flow at the ostium and thus minimizes undesirable reactions such as those listed above.

### BRIEF SUMMARY OF THE INVENTION

Embodiments of the present invention are directed to an intraluminal stent system for placement in a bifurcated vessel. The stent system includes a side branch stent for placement in the side branch vessel. The side branch stent includes an expandable metallic distal section and a proximal section. A distal end of the proximal section is coupled to the distal section. The proximal section is made from a flexible material in the form of a sheet and is not supported by a frame proximally of the distal end. A proximal end of the proximal section is curled or tabbed to engage walls of the main vessel at a junction of the main vessel and the side branch vessel. A main branch stent includes an expandable body portion having a generally cylindrical hollow shape with an outer diameter. The main branch stent is configured to push the curled or tabbed proximal end of the first stent against the walls of the main vessel at the junction. The side branch stent and main branch stent may be balloon expandable or self expandable.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a schematic illustration of a side branch stent in accordance with an embodiment of the present invention.

FIG. 1A is a schematic illustration of a side branch stent in accordance with another embodiment of the present invention.

FIG. 2 is illustrates a portion of a vessel at a bifurcation with a guidewire advanced into the side branch vessel.

FIG. 3 illustrates the vessel of FIG. 2 with a catheter advanced into the side branch vessel and with the stent of FIG. 1 mounting on the catheter.

FIG. 4 illustrates the catheter of FIG. 3 with the balloon inflated to expand the distal section of the stent of FIG. 1.

FIG. 5 illustrates the side branch stent of FIG. 1 in place in the side branch vessel after the catheter has been removed.

FIG. 6 illustrates a second catheter advanced into the main vessel of the bifurcated vessel.

FIG. 7 illustrates the catheter FIG. 6 subsequent to inflation of the balloon.

FIG. 8 illustrates the side branch stent of FIG. 1 in place in the side branch vessel and the main vessel stent in place in the main vessel after the second catheter of FIG. 6 has been removed.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful, including any body lumen or tubular tissue within the cardiac, coronary, renal, peripheral vascular, gastrointestinal, pulmonary, urinary and neurovascular systems and the brain. Furthermore, there is no intention to be bound by any expressed or implied theory presented in the preceding technical field, background, brief summary or the following detailed description.

In accordance with embodiments of the present invention, a stent has a generally cylindrical hollow body portion and is intended for placement in a side branch vessel associated with a bifurcated or trifurcated vessel. The side branch stent includes a proximal section formed of a flexible membrane and a distal section formed of a metallic material such as a conventional stent. The flexible membrane of the proximal section is not supported by a stent frame. The proximal section may be flared or curled such that upon expansion of the side branch stent, the flared or curled end may extend into the main branch vessel of a bifurcated vessel.

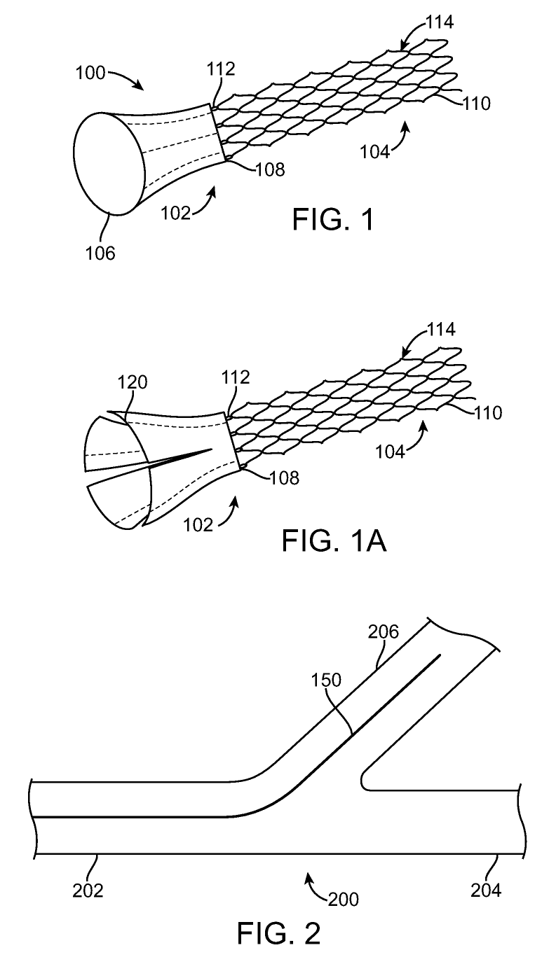

FIG. 1 illustrates a side branch stent 100 intended for placement in a side branch vessel associated with a bifurcated or trifurcated vessel. Side branch stent 100 includes a proximal section 102 and a distal section 104. Proximal section 102 is made from a flexible graft material such as Dacron (polyester), polytetrafluoroethylene (“PTFE”), expanded polytetrafluoroethylene (“ePTFE”), nylon, or other synthetic arteriovenous access graft materials. Proximal section 102 does not include a frame supporting the flexible graft material. As can be seen in FIG. 1, the flexible graft material of proximal section 102 is in the form of a sheet. Proximal section 102 includes a flared or curled proximal end 106 that is designed to extend into the main vessel of a bifurcated or trifurcated vessel. Proximal end 106 may alternatively include tabs 120 as shown in FIG. 1A. Distal section 104 is a conventional metallic stent. Forming proximal section of a flexible graft material provides a more laminar flow into the side branch vessel to reduce the possibility of restenosis. Further, using a flexible graft material for the proximal section 102 of side branch stent 100 provides less metallic restenosis initiation sites in the path of the blood flow, also reducing the possibility of restenosis.

In the particular embodiment shown, distal section 104 includes a plurality of stent struts 110 formed into a generally hollow cylindrical configuration. One configuration for stent struts 110 includes a plurality of undulating or wavelike bands 114 having straight segments and turns (i.e., alternating turns facing opposite longitudinal directions). Bands 114 are aligned on a common longitudinal axis to form a generally cylindrical body having a radial and longitudinal axis and are connected together to form distal section 104. As will be apparent to those of ordinary skill in the art, stent struts 110 may have any suitable pattern, such as for example, a cross-hatched pattern, mesh pattern, or a coiled pattern. Distal section 104 may be any stent body known in the art may that has a suitable generally cylindrical configuration such as the stents shown or described in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau, which are incorporated by reference herein in their entirety.

Distal section 104 may be made from a variety of medical implantable metallic materials, including, but not limited to, stainless steel, nickel-titanium (nitinol), cobalt-chromium, tantalum, ceramic, nickel, titanium, aluminum, nickel-cobalt alloy such as MP35N, titanium ASTM F63-83 Grade 1, niobium, platinum, gold, silver, palladium, iridium, combinations of the above, and the like. Once implanted, the distal section 104 provides artificial radial support to the wall tissue of a side branch vessel. In one embodiment of the present invention, at least a portion of distal section 104 of side branch stent 100 may be coated with a therapeutic agent (not shown). The therapeutic agent can be of the type that dissolves plaque material forming the stenosis or can be such as an antineoplastic agent, an antiproliferative agent, an antibiotic, an antithrombogenic agent, an anticoagulant, an antiplatelet agent, an anti-inflammatory agent, combinations of the above, and the like.

Proximal section 102 and distal section 104 are joined at junction 108 by, for example, loops 112 coupling the flexible material of proximal section 102 to struts of distal section 104. Other suitable means for couple proximal section 102 and distal section 104 may be used, as would be apparent to those of ordinary skill in the art.

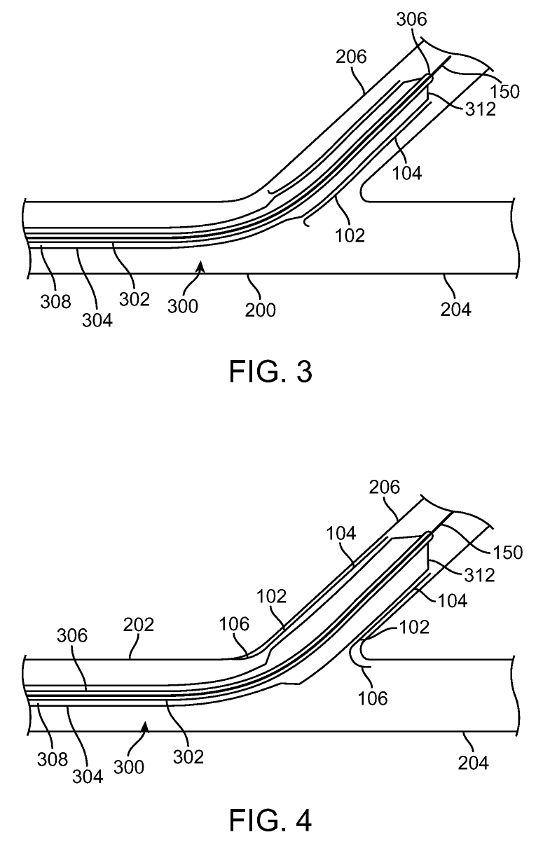

Proximal section 102 and distal section 104 may be self-expandable or balloon expandable. With reference to FIGS. 2-8, an embodiment of a method for delivering side branch stent 100 to a side branch vessel 206 will now be discussed. A vessel 200 includes a main vessel 202, a main branch vessel 204, and side branch vessel 206. A guidewire 150 is tracked through main vessel 202 and into side branch vessel 206, as illustrated in FIG. 2. A catheter assembly 300 is tracked over guidewire 150 and into side branch vessel 206. In one embodiment, the catheter assembly 300 includes an inner shaft 302, an outer shaft 304, and a balloon 312. Inner shaft 302 includes a guidewire lumen 306, through which guidewire 150 is disposed to track catheter assembly 300 over guidewire 150. Balloon 312 is attached at a proximal end to outer shaft 304 and at a distal end to inner shaft 302. An inflation lumen 308 is disposed between inner shaft 302 and outer shaft 304, and opens into an interior space of balloon 312. Side branch stent 100 is disposed over balloon 312. In an alternative embodiment (not shown) wherein side branch stent 100 is self-expandable, the catheter assembly need not include a balloon 312. Instead, a sheath covers side branch stent 100 and is withdrawn to allow side branch stent 100 to self-expand. Catheter assembly 300 is a conventional over-the-wire catheter. As would be understood by those of ordinary skill in the art, other catheter assemblies could be used, such as rapid-exchange catheters.

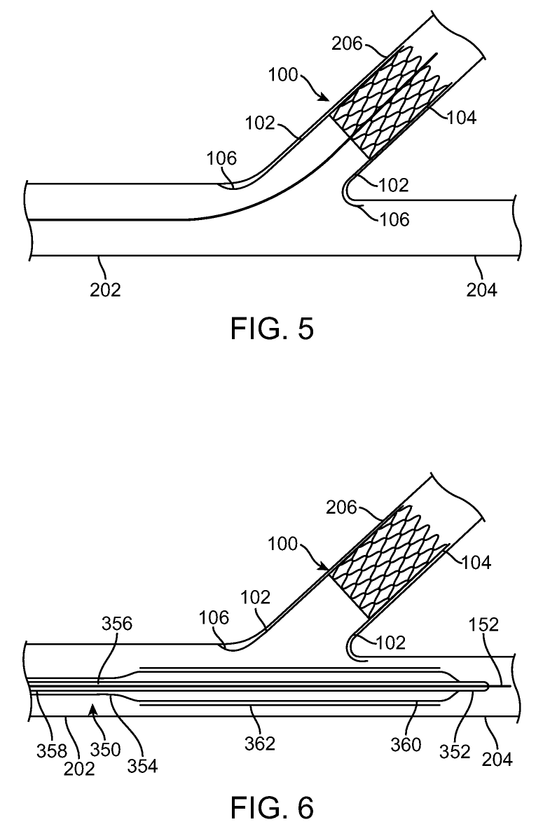

After catheter assembly 300 has been tracked into side branch vessel 206, inflation fluid is injection into balloon 312 through inflation lumen 308, thereby expanding balloon 312 and consequently, distal section 104 and proximal section of side branch stent 100, as shown in FIG. 4. Inflation fluid is then drained from inflation lumen 308 and balloon 312, thereby deflating balloon 312. In an alternative embodiment (not shown), a sheath covering side branch stent 100 is removed to allow side branch stent 100 to self-expand. Catheter assembly 300 is then removed proximally, leaving side branch stent 100 deployed in side branch vessel 206, as shown in FIG. 5. As can be seen in FIG. 5, curled proximal end 106 of proximal section 102 extends into main vessel 202.

After balloon 312 has been deflated and catheter assembly 300 has been removed from the bifurcation area, a second guidewire 152 is through main vessel 202 and into main vessel branch 204. In the alternative, guidewire 150 may be withdrawn out of side branch vessel 206 and redirected into main vessel branch 204. A second catheter assembly 350 is advanced over second guidewire 152, as shown in FIG. 6, to the bifurcation site. Second catheter assembly 350 in this embodiment includes an inner shaft 352, an outer shaft 354, and a balloon 360. A guidewire lumen 356 is disposed within inner shaft 352 to track second catheter 350 over second guidewire 152. An inflation lumen 358 is disposed between inner shaft 352 and outer shaft 354, and opens into an interior of balloon 360. A main vessel stent 362 is disposed around balloon 360.

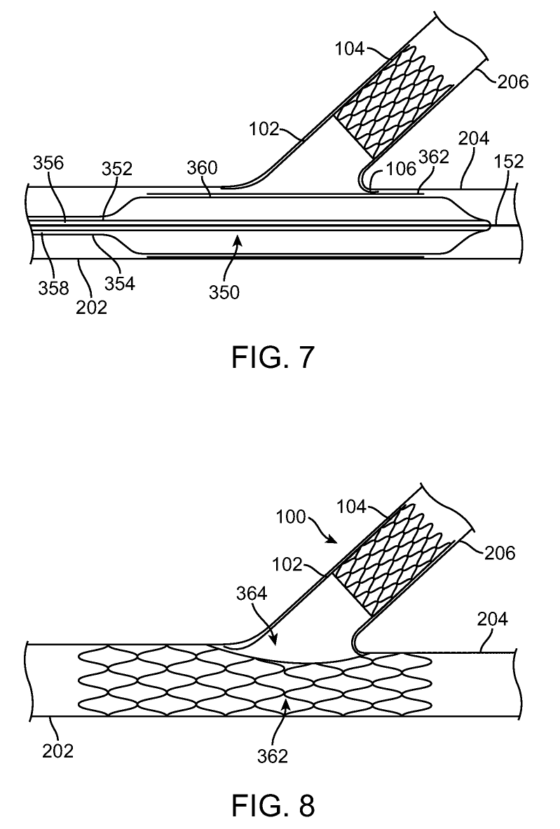

After second catheter assembly 350 has been tracked to the bifurcation site, inflation fluid is injection into balloon 360 through inflation lumen 358, thereby expanding balloon 360 and consequently, main vessel stent 362, as shown in FIG. 7. Inflation fluid is then drained from inflation lumen 358 and balloon 360, thereby deflating balloon 360. Second catheter assembly 350 is then removed proximally, leaving main vessel stent 362 deployed in main vessel 202 and main branch vessel 204, as shown in FIG. 8. As can be seen in FIG. 8, pressure from balloon 360 and main vessel stent 362 pushes curled proximal end 106 of side branch stent 100 against the vessel walls at the bifurcation such that curled proximal end 106 of proximal section 102 apposes the vessel walls.

Main vessel stent 362 may be made from a variety of medical implantable materials, including, but not limited to, stainless steel, nickel-titanium (nitinol), cobalt-chromium, tantalum, ceramic, nickel, titanium, aluminum, polymeric materials, nickel-cobalt alloy such as MP35N, titanium ASTM F63-83 Grade 1, niobium, platinum, gold, silver, palladium, iridium, combinations of the above, and the like. In various embodiments of the present invention, the main vessel stent may be made from a metallic material. Once implanted, main vessel stent 362 provides artificial radial support to the wall tissue. Further, at least a portion of the main vessel stent may be coated with a therapeutic agent (not shown). The therapeutic agent can be of the type that dissolves plaque material forming the stenosis or can be such as an antineoplastic agent, an antiproliferative agent, an antibiotic, an antithrombogenic agent, an anticoagulant, an antiplatelet agent, an anti-inflammatory agent, combinations of the above, and the like.

Main vessel stent 362 may be formed using any of a number of different methods. For example, the main vessel stent may be formed by winding a wire or ribbon around a mandrel to form a strut pattern like those described above and then welding or otherwise mechanically connecting two ends thereof to form a circular band. A plurality of circular bands are subsequently connected together to form the main vessel stent. Alternatively, main vessel stent 362 may be manufactured by machining tubing or solid stock material into toroid bands, and then bending the bands on a mandrel to form the pattern described above. A plurality of circular bands formed in this manner are subsequently connected together to form the longitudinal stent body. Laser or chemical etching or another method of cutting a desired shape out of a solid stock material or tubing may also be used to form the main vessel stent of the present invention. In this manner, a plurality of circular bands may be formed connected together such that the stent body is a unitary structure. Further, main vessel stent 362 may be manufactured in any other method that would be apparent to one skilled in the art. The cross-sectional shape of the main vessel stent may be circular, ellipsoidal, rectangular, hexagonal rectangular, square, or other polygon, although at present it is believed that circular or ellipsoidal may be preferable.

As shown in FIG. 8, main vessel stent may include an opening 364 that is placed adjacent to the opening from the main vessel 202 to side branch vessel 206. Opening 364 provides less interference and turbulence of the flow of blood into side branch vessel 206. Alternatively, a cell (that is, the space between the struts of main vessel stent 362) adjacent to the opening to side branch vessel 206 may be expanded such that struts of main vessel stent do not interfere with flow into the side branch vessel.

Although side branch stent 100 and main vessel stent 362 have been described as balloon expandable stents, it would be apparent to those of ordinary skill in the art that either or both can be self-expandable stents. In the case of side branch stent 100, a sheath would cover both proximal section 102 and distal section 104. After reaching the proper location in side branch vessel 206, the sheath is retracted proximally and distal section 104 and proximal section 102 expand to their expanded configurations. Similarly, main vessel stent 362 can be self-expandable, and balloon 360 would be replaced by a sheath to keep main vessel stent 362 in the contracted configuration. Upon reaching the desired location, the sheath would be retracted, allowing main vessel stent 362 to expand, as known to those of ordinary skill in the art.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An intraluminal stent device for placement in a side branch vessel associated with a bifurcated vessel, comprising:
an expandable metallic distal section having a generally cylindrical hollow shape; and
a proximal section, wherein a distal end of said proximal section is coupled to said distal section, wherein said proximal section is made from a flexible material in the form of a sheet and is not supported by a frame proximally of said distal end, and wherein said proximal section further includes a proximal end adapted to engage walls of a main vessel at a junction of the main vessel and the side branch vessel.

2. The intraluminal stent device of claim 1, wherein said flexible material is selected from the group consisting of polyester, polytetrafluoroethylene, expanded polytetrafluoroethylene, and nylon.

3. The intraluminal stent device of claim 1, wherein said distal section has a first unexpanded outer diameter and a second expanded outer diameter, and wherein said proximal section has a first unexpanded outer diameter and a second expanded outer diameter.

4. The intraluminal stent device of claim 1, wherein said proximal section and said distal section are balloon expandable.

5. The intraluminal stent device of claim 1, wherein said proximal section and said distal section are self expandable.

6. The intraluminal stent device of claim 1, wherein said distal section made of a material selected from the group consisting of stainless steel, nickel-titanium, cobalt-chromium, tantalum, ceramic, nickel, titanium, aluminum, nickel-cobalt alloy, titanium, niobium, platinum, gold, silver, palladium, iridium, and combinations thereof.

7. The intraluminal stent device of claim 1, wherein the proximal end of said proximal section is curled to engage the walls of the main vessel at the junction of the main vessel and the side branch vessel.

8. The intraluminal stent device of claim 1, wherein the proximal end of said proximal section includes tabs adapted to fold to engage the walls of the main vessel at the junction of the main vessel and the side branch vessel.

9. An intraluminal stent system for placement in a bifurcated vessel including a main vessel, a main vessel branch, and a side branch vessel, the stent system comprising:
a first stent configured for placement in the side branch vessel, the first stent having an expandable metallic distal section and a proximal section, wherein a distal end of the proximal section is coupled to the distal section, the proximal section is made from a flexible material in the form of a sheet and is not supported by a frame proximally of the distal end, and the proximal section further includes a proximal end adapted to engage walls of the main vessel at a junction of the main vessel and the side branch vessel; and
a second stent configured for placement in the main vessel and the main vessel branch, the second stent including an expandable body portion having a generally cylindrical hollow shape with an outer diameter, wherein the second stent is configured to push the proximal end of the first stent against the walls of the main vessel at the junction.

10. The intraluminal stent system of claim 9, wherein the second stent includes an opening aligned with the junction.

11. The intraluminal stent system of claim 9, wherein said flexible material is selected from the group consisting of polyester, polytetrafluoroethylene, expanded polytetrafluoroethylene, and nylon.

12. The intraluminal stent system of claim 9, wherein the distal section of said first stent has a first unexpanded outer diameter and a second expanded outer diameter, and wherein the proximal section of said first stent has a first unexpanded outer diameter and a second expanded outer diameter.

13. The intraluminal stent system of claim 9, wherein the proximal section and the distal section of said first stent are balloon expandable.

14. The intraluminal stent system of claim 9, wherein the proximal section and the distal section of said first stent are self expandable.

15. The intraluminal stent system of claim 9, wherein the distal section of said first stent is made of a material selected from the group consisting of stainless steel, nickel-titanium, cobalt-chromium, tantalum, ceramic, nickel, titanium, aluminum, nickel-cobalt alloy, titanium, niobium, platinum, gold, silver, palladium, iridium, and combination thereof.

16. The intraluminal stent system of claim 9, wherein said second stent is self-expandable.

17. The intraluminal stent system of claim 9, wherein said second stent is balloon expandable.

18. The intraluminal stent system of claim 9, wherein said second stent is made of a material selected from the group consisting of stainless steel, nickel-titanium, cobalt-chromium, tantalum, ceramic, nickel, titanium, aluminum, polymeric materials, nickel-cobalt alloy, titanium, niobium, platinum, gold, silver, palladium, iridium, and combinations thereof.

19. The intraluminal stent system of claim 9, wherein the proximal end of the proximal section of said first stent is curled to engage the walls of the main vessel at the junction of the main vessel and the side branch vessel.

20. The intraluminal stent system of claim 9, wherein the proximal end of the proximal section of said first stent includes tabs adapted to fold to engage the walls of the main vessel at the junction of the main vessel and the side branch vessel.

21. A method of placing an intraluminal stent system in a bifurcated vessel having a main vessel and a side branch vessel, the method comprising the steps of:
providing a first stent configured for placement in the side branch vessel, wherein the first stent is expandable from a collapsed configuration to an expanded configuration, the first stent including metallic distal section and a proximal section, wherein a distal end of the proximal section is coupled to the distal section, wherein the proximal section is made from a flexible material in the form of a sheet and is not supported by a frame proximally of the distal end, and wherein the proximal section further includes a proximal end adapted to extend outside of the side branch vessel and engage walls of the main vessel at a junction of the main vessel and the side branch vessel;
providing a second stent configured for placement in the main vessel, wherein the second stent is expandable from a collapsed configuration to an expanded configuration, the second stent having a plurality of struts with cells or spaces there between and forming a generally cylindrical hollow shape with an outer surface;
delivering the first stent to the side branch vessel of the bifurcated vessel and positioning the first stent such that the curled proximal end extends outside of the side branch vessel into the main vessel;
deploying the first stent in the side branch vessel;
inserting the second stent in the collapsed configuration into the main vessel such that a portion of the outer surface of the second stent extends over the proximal end of the first stent;
deploying the second stent in the main vessel such that the outer surface of the second stent pushes the proximal end of the first stent against the walls of the main vessel at the junction.

22. The method of claim 21, wherein the step of deploying the first stent in the side branch vessel includes inflating a balloon of a balloon catheter to expand the first stent.

23. The method of claim 21, wherein the proximal section and the distal section of the first stent are self-expandable such that the step of deploying the first stent in the side branch vessel includes removing a sheath covering the proximal section and the distal section.

24. The method of claim 21, wherein the step of deploying the second stent in the main vessel includes inflating a balloon of a balloon catheter.

25. The method of claim 21, wherein the second stent is self-expandable such that the step of deploying the second stent in the main vessel includes remove a sheath covering the second stent.

26. The method of claim 21, wherein the distal section of the first stent is made of a material selected from the group consisting of stainless steel, nickel-titanium, cobalt-chromium, tantalum, ceramic, nickel, titanium, aluminum, nickel-cobalt alloy, titanium, niobium, platinum, gold, silver, palladium, iridium, and combination thereof.

27. The method of claim 21, wherein the flexible material of the proximal section of the first stent is selected from the group consisting of polyester, polytetrafluoroethylene, expanded polytetrafluoroethylene, and nylon.

28. The method of claim 21, wherein the second stent is made of a material selected from the group consisting of stainless steel, nickel-titanium, cobalt-chromium, tantalum, ceramic, nickel, titanium, aluminum, polymeric materials, nickel-cobalt alloy, titanium, niobium, platinum, gold, silver, palladium, iridium, and combinations thereof.

29. The method of claim 21, wherein the proximal end of the proximal section of said first stent is curled to engage the walls of the main vessel at the junction of the main vessel and the side branch vessel.

30. The method of claim 21, wherein the proximal end of the proximal section of said first stent includes tabs adapted to fold to engage the walls of the main vessel at the junction of the main vessel and the side branch vessel.
